(19) United States
(12) Patent Application Publication
(10) Pub. No.: US 2009/0029408 A1
(43) Pub. Date: Jan. 29, 2009

(54) Methods for Characterizing Cell Proximity

(75) Inventors: Peter LaPan, Arlington, MA (US); Steven A. Haney, Concord, MA (US)

Correspondence Address: CHOATE, HALL & STEWART LLP/WYETH, PATENT GROUP, TWO INTERNATIONAL PLACE, BOSTON, MA, 02110, US

(73) Assignee: WYETH, Madison, NJ (US)

(21) Appl. No.: 12/179,245

(22) Filed: Jul. 24, 2008

Related U.S. Application Data
(60) Provisional application No. 60/961,886, filed on Jul. 25, 2007.

(51) Int. Cl. C12Q 1/04

(52) U.S. Cl. 435/34

(57) ABSTRACT

One aspect of the invention relates to a method of identifying cell adjacency in a coculture of cells. The method includes generating a first set of image data of the coculture of cells, the first set of image data comprising nuclear position data for each imaged cell; generating a second set of image data of at least a subset of the coculture of cells, the second set of image data comprising cell identifier data associated with a first cell type; generating a third set of image data of at least a subset of the coculture of cells, the third set of image data comprising cell identifier data associated with a second cell type; and identifying a group of cells in response to the nuclear position data, wherein each cell having the first cell type in the group is adjacent another cell in the group having the second cell type.

### RELATED APPLICATIONS

This application claims priority under 35 U.S.C. § 119(e) to U.S. Provisional Application U.S. Ser. No. 60/961,886 filed on Jul. 25, 2007, which is incorporated herein by reference in its entirety.

### FIELD OF THE INVENTION

In general, aspects of the invention relate to methods and apparatus for distinguishing populations and sub-populations of cells. In particular, adjacent cells can be distinguished from non-adjacent cells in a coculture using the techniques disclosed herein.

### BACKGROUND OF THE INVENTION

Cells often have numerous chemical compounds, such as proteins and polysaccharides, and other structural features integrated within their cellular membrane or cell walls. These compounds and structures can exhibit various degrees of activity during the lifecycle of a cell. However, the proximity of one cell to another can cause changes in one or both cells. This follows because the chemical compounds and structural features discussed above allow a cell to influence and be influenced by its environment.

For example, the proximity of multiple cells during a developmental event may cause chemicals to be exchanged between the cells that initiate vascular development or cause controlled cell death. Of particular interest to many researchers is the ability to identify a disease pathology such as tumor development or plaque formation. To the extent that extra-cellular interactions are correlated to these pathologies, identifying them is of interest to the scientific community. Unfortunately, an efficient approach for performing such investigations as part of a large scale, high speed process has not been available.

Identifying and taking measurements on a subpopulation of cells allows for the collection of data from this subpopulation of cells. These responses may otherwise be statistically insignificant if averaged into the total population of cells. Therefore, there is a need for methods and apparatus that facilitate studying the interactions between two or more cells and selective identification of subpopulations within other large cell populations, such as cocultures.

### SUMMARY OF THE INVENTION

In part, aspects of the present invention address a need for methods and apparatus that enable detection of cell adjacency and characterization and evaluation of two or more cells' geometric proximity across different cell types. Specifically, one aspect of the present invention relates to a method of identifying, in a coculture of cells of differing cell types, cells of a first cell type in contact with cells of a second cell type. The method includes distinguishing cells of a first cell type from cells of a second cell type and identifying cells of a first cell type in contact with cells of a second cell type. The process of distinguishing cells may include labeling the cells of a first cell type with a first label to distinguish cells of the first cell type from the cells of the second cell type, detecting the first label to identify cells of the first cell type, labeling the cells of the second cell type, but not the first cell type, with a second label and detecting the second label to facilitate distinguishing cells of the first cell type from the cells of the second cell type. The process of identifying cells may include identifying cells labeled with the first label adjacent to cells labeled with the second label or labeling the nuclei of the cells of the first cell type and the cells of the second cell type with a nuclear label, detecting the nuclear label, thereby to detect the locations of the nuclei in the coculture, identifying cells having internuclear distances less than or equal to approximately one cell diameter, thereby to identify cells in contact with each other and/or calculating distances between nuclei in the coculture. In some embodiments, the second label is a labeled anti-CD44 antibody.

In some embodiments, the first cell type is an endothelial cell and the second cell type is an epithelial cell. In this case, distinguishing cells includes labeling the epithelial cells, but not the endothelial cells, with a labeled anti-CDW75 antibody and detecting the labeled anti-CDW 75 antibody. In some embodiments, the method of identifying a cancer epithelial cell includes exposing the cancer epithelial cell to an anti-CDW75 antibody and detecting the anti-CDW 75 antibody to identify the cancer epithelial cell.

Another embodiment of the present invention relates to a method of identifying subpopulations of cells within a population of cells. The method includes assigning a spatial coordinate to each cell in the population, the spatial coordinate identifying a relative position of each cell; identifying a first subpopulation of the population using a distance metric, each cell of the first subpopulation substantially adjacent to another cell in the population; and identifying a second subpopulation of the first subpopulation using a cell characteristic, each cell of the second subpopulation substantially adjacent to a cell of interest, the cell of interest having a cell type. The distance metric may relate two spatial coordinates and cell diameter data to determine cell adjacency. In one embodiment, the cell characteristic is selected from the group consisting of antibody affinity, receptivity to a stain, receptivity to a label, light reflectance, fluorescence, and light absorption. The cell type may be selected from the group consisting of endothelial, epithelial, stem, and cancer.

Another embodiment of the present invention relates to a method of identifying cell adjacency in a coculture of cells. The method includes generating a first set of image data of the coculture of cells, the first set of image data comprising nuclear position data for each imaged cell; generating a second set of image data of at least a subset of the coculture of cells, the second set of image data comprising cell identifier data associated with a first cell type; generating a third set of image data of at least a subset of the coculture of cells, the third set of image data comprising cell identifier data associated with a second cell type; and identifying a group of cells in response to the nuclear position data, wherein each cell having the first cell type in the group is adjacent another cell in the group having the second cell type. In one embodiment the coculture of cells is disposed in a well of a multi-well plate. The first cell type may be endothelial and the second cell type is epithelial. The cell identifier data may be generated in response to light originating from a labeled cell.

Aspects of the invention improve on neighbor detection strategies by allowing detection of neighbors of a different cell type across fluorescent channels. Aspects of the invention also flag objects as adjacent or nonadjacent, allowing for quantification and comparison of features across the two cell populations.

Other features, objects, and advantages of the present invention are apparent in the detailed description that follows. It should be understood, however, that the detailed description, while indicating embodiments of the present invention, is given by way of illustration only, not limitation. Various changes and modifications within the scope of the invention will become apparent to those skilled in the art from the detailed description.

### BRIEF DESCRIPTION OF THE DRAWINGS

FIG. 1 is a high level flow chart depicting a method for characterizing cells in a coculture according to an illustrative embodiment of the invention.

FIG. 2 is a depiction of a cell oriented coordinate system and distance metric calculation method according to an illustrative embodiment of the invention.

FIG. 3 is a high level flow chart depicting another exemplary measurement technique according to an illustrative embodiment of the invention.

FIG. 4 is a high level flow chart depicting yet another measurement technique according to an illustrative embodiment of the invention.

FIG. 5A is a depiction of a coculture of cells labeled using a fluorescent stain according to an illustrative embodiment of the invention.

FIG. 5B is a depiction of coculture of cancer cells labeled using an antibody according to an illustrative embodiment of the invention.

FIG. 5C is a scatter plot of the x-y coordinates of each cell type in the cell coculture as obtained in accordance with an illustrative embodiment of the invention.

Various aspects of the invention are described in further detail in the following subsections. The use of subsections is not meant to limit the invention. Each subsection may apply to any aspect of the invention. In this application, the use of “or” means “and/or” unless stated otherwise. As used in this disclosure, the term “comprise” and variations of the term, such as “comprising” and “comprises,” are not intended to exclude other additives, components, integers or steps. For all of the methods described herein, one or more of the method steps or a result thereof may be displayed to a user via an output device to produce a useful and tangible result.

### DETAILED DESCRIPTION

Currently, there is evidence for both the stimulatory and inhibitory effects of cancer epithelial cells (i.e. the cancer cells that line the inside and outside of the human body) on endothelial cells (i.e., thin, flattened cell which line the inside surfaces of body cavities, blood vessels, and lymph vessels) proliferation and differentiation. The cancer cells respond to endothelial cell contact by secreting growth factors that stimulate endothelial cell proliferation and migration. Cancer cells have also been reported to induce apoptosis (i.e. programmed cell death or cell suicide) of neighboring endothelial cells, a process that is thought to impair vascular integrity, enhance extravasation (i.e. the process whereby a cancer cell exits a blood vessel or lymphatic vessel), and promote metastasis (i.e. the migration of cancer cells from the original tumor site through the blood and lymph vessels to produce cancers in other tissues) in vivo.

Elucidation of the function of various gene products (i.e. potential drug targets) requires sensitive assays that can measure responses in an often minor, spatially restricted subpopulation of cells. The ability to collect measurements from single cells adjacent to cancer epithelial cells, and compare these measurements to control cells that are not in contact with cancer epithelial cells will allow the assessment of the impact of cancer cell contact on endothelial cell proliferation, migration, and differentiation. Pathways and mechanisms responsible for the effects of cancer epithelial cells on endothelial cells can be investigated using small molecules or siRNAs. The aspects of the invention described herein can accomplish these goals and objectives.

Specifically, the present invention relates generally to a system and method for identifying different subpopulations of cells (adjacent and non-adjacent) as a function of cell type. Aspects of the invention make use of a general nuclear label, cell type specific surface labels, image capture hardware, image analysis software, and distance-based and statistical algorithms. These elements can be integrated using the techniques outlined herein to identify adjacent cells in cocultures of different cell types, such as endothelial cells and epithelial cells.

In particular, aspects of the invention can be used to identify populations of cancer cells that are adjacent to populations of non-cancer cells in order that compounds and other treatment regimens can be systematically evaluated. Since a cancer cell can promote growth in other cells, such as initiating vascularization which nourishes the cancer cell, it is desirable to identify drugs that will inhibit the chemical and genetic signals transmitted by cancer cells to adjacent cells. As a result, it desirable to identify instances of adjacency and non-adjacency between cancer cells, and other cell types, in a mixed population.

However, the mixed population of cell types considered herein can include more than two cell types; in a preferred embodiment the techniques described herein are adapted for identifying cell adjacency and non-adjacency between cells in a coculture that includes two cell types. A coculture is a collection of distinct cell types in a combined growth media. The coculture may be contained in a beaker, a slide, a plate or a multi-well plate. As described herein in more detail below, a multi-well plate embodiment is useful for research purposes as the effects of drugs and other factors can be evaluated using a batch process with a plurality of cocultures resident in different wells on the plate. The cells resident in the coculture may be any cells of interest, for example, an endothelial cell of an animal, for example, a human cell, an epithelial cell, a cancerous cell or any other cell type of interest.

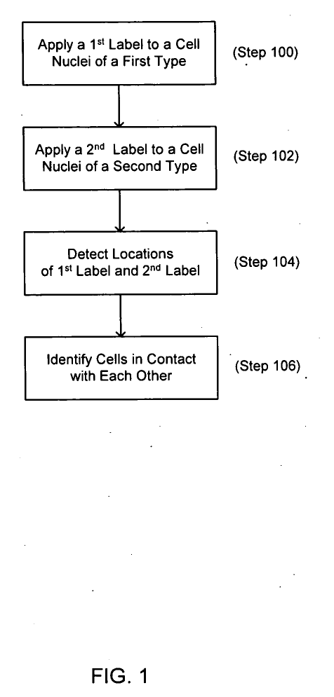

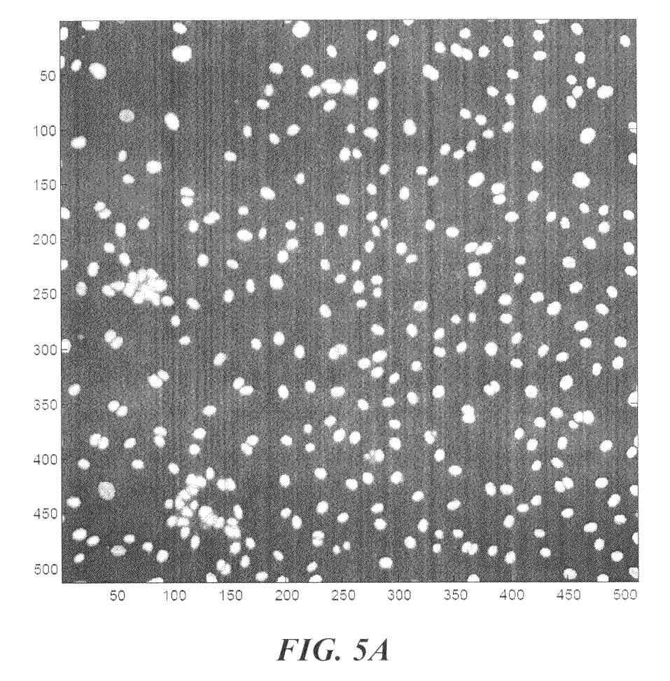

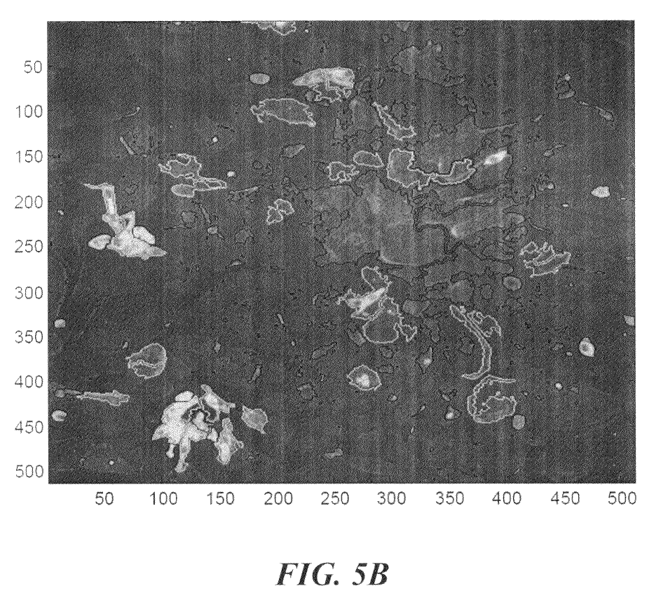

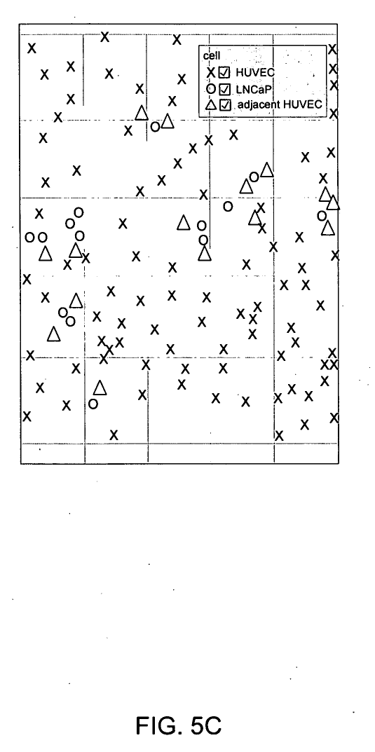

FIG. 1 is a high level flow chart that depicts an exemplary embodiment of a method for determining subpopulations of adjacent and non-adjacent cells as function of cell type. In one preferred embodiment, three different labels are used, one general nuclear stain, and two cell type specific surface stains are used in accordance with this method. Initially, a nuclear stain is applied to the overall population of mixed cell types in the coculture (Step 100). As discussed in more detail below, the nuclear stain advantageously allows for all of the cells in the coculture to be identified and nuclear coordinates to be assigned on a per nuclei basis. Thus, the relative positions of all of the nuclei and their associated cells can be determined. An example of an image showing stained nuclei is shown in FIG. 5A. In one embodiment, DAPI is used as the nuclear stain. DAPI or 4′,6-diamidino-2-phenylindole is a fluorescent stain that binds strongly to DNA. It is a highly specific and sensitive fluorescing DNA stain used in epifluorescent microscopy to observe structures containing DNA. However, other nuclear stains can be used as is known in the relevant arts.

As shown in FIG. 1, after the application of the general nuclear stain, the next step is to apply a first label to cells of a first type (Step 101). Typically, this first label is a surface label that can be used to identify all of the cells in the coculture of a first particular cell type. Thus, a first label may be applied to a mixed population of cell types that selectively and exclusively binds to one cell type, for example, all of the epithelial cells in the population can be so labeled.

Still referring to FIG. 1, as a next step, a second label is applied to the nuclei of a subpopulation of cells of a second type (Step 102). The second label may be similar to the first label applied to the first subpopulation of cells, but the second label is selected such that it will bind to cells of a second cell type. Thus, the selection of the first and second labels is made such that cells of a first cell type can be distinguished from cells of second cell type within an overall coculture population of mixed first and second cell types.

The label may be any marker that can be used to identify and distinguish cells given the previous application of a general nuclear stain. For example, in a mixture of endothelial cells, cancerous epithelial cells, regular epithelial cells, and various other particulate matter and cells, the first label will only react with the endothelial cells and not the other cell types. In one embodiment, the label may be a fluorescent cell type specific dye, which may include a cell marker, for example an antibody such as CD44 (a cell surface glycoprotein found on HUVEC (endothelial) cells) or CDw75 (an alpha 2,6-sialylated carbohydrate molecule that is found on LNCaP cells (epithelial prostate cancer cells)) and a florescent molecule, such as Alexa 488 and Alexa 594.

In another embodiment, the same stain or label may be used for multiple types of cells. In this embodiment, the cells may be distinguished later in the detection process as function of the degree, gradient, or intensity of the staining, i.e., one cell type may absorb more of the stain than another cell type. As a result, the detector can be calibrated to detect varying degrees of staining and the associated intensity. In turn, the previous application of the general nuclear stain allows coordinate positions to be assigned to all of the cells in the coculture. This assignment of coordinate positions further facilitates the assessment of the relative positions of different cells as a function of the known average cell diameter for the first cell type and the second cell type.

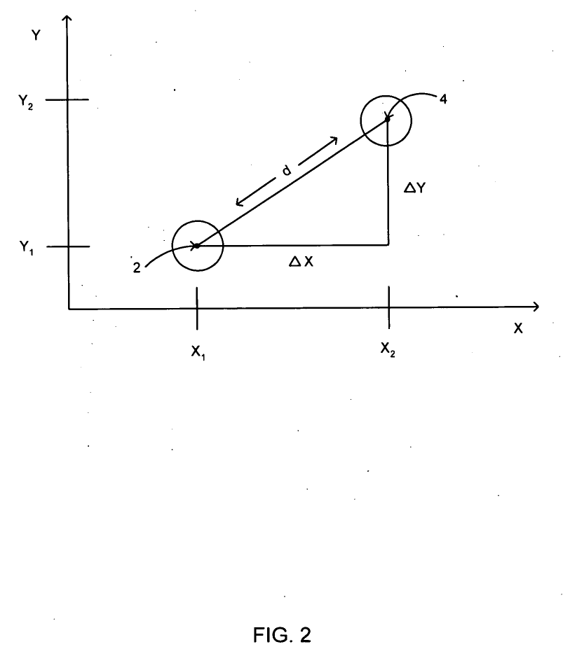

A high content image system such as a fluorescent microscopy system with a CCD detector can be used to detect the three different labels discussed above. In one embodiment, the image data is analyzed through the use of high content image analysis software, for example Cellomics' Arrayscan Bioapplication (Cellomics, Inc. 100 Technology Drive, Pittsburgh, Pa. USA 15219) or the open source CellProfiler image analysis software (Broad Institute, 7 Cambridge Center Cambridge, Mass. 02142). This software identifies and outlines the nuclei of all the cells present in a coculture. The software provides the x-y coordinate of the different nuclei within an image, as shown in FIG. 2.

Using the information generated from the staining and labeling, the next step is to identify cells in contact (adjacent or non-adjacent) with each other (Step 104). The general concept is that each cell of the first type and each cell of the second type have average cell diameters, respectively. In one embodiment, the cell diameter of both cell types is assumed to be substantially the same. However, variations in cell diameter can be incorporated into the operation of the distance metric or by performing iterative comparisons using the different cell diameters with the same metric.

Given the (x, y) position information for all of the cells in the coculture, each cell in the coculture can be iteratively compared to every other cell in the coculture to ascertain the relative distances between all of the cells. If the neighboring distance between two cells is less than the average cell diameter specified, those two cells can be scored as adjacent. In contrast, if two cells have a neighboring distance that exceeds the average cell diameter specified, than those two cells would be scored as non-adjacent by the software performing the calculation. By performing this analysis using Boolean logic or other algorithmic comparative approaches, all of the cells in the coculture can be scored as adjacent or non-adjacent using a distance metric. As a result, as discussed below, the next step is to use the information from the other surface labels to determine which cells are both adjacent and of different cell types. Specific details relating to the use of distance metric to perform the comparison and scoring of cells given their geometric proximity are described with respect to FIG. 2.

In one embodiment, the R software environment (R Foundation for Statistical Computing; Department for Statistics and Mathematics; Wirtschaftsuniversit\ddot{} at Wien, Augasse 2-6, 1090 Wien, Austria) is programmed to perform this and other calculations using an R script that operates on a dataset such as the partial dataset recited below as Table 1. An exemplary R script is provided herewith as TABLE 2. However, the principles and techniques described herein can be performed using any suitable programming environment and data set of any suitable computer readable format. Thus, in no way are TABLE 2, Table 1, or the use of any of the apparatus and software described herein limiting as to the scope of the aspects of the invention. Further, although various images are depicted with respect to FIGS. 5A-5C, the calculations described herein are typically performed using raw captured data or a formatted data file without using a geometric image to perform any calculations.

Identifying the nuclear positions used above can be achieved using an optical detection system or other label detection mechanisms as known in the art to obtain image data. Initially, the detection mechanism captures image data that includes an optical channel suitable to receive wavelengths associated with the nuclear staining of substantially all of the cells in the population. The detector may be a fluorescent detector, a CCD camera, an inverted fluorescent microscope with a CCD camera, or any other device known to one of skill in the art for detecting photons. The detector captures image data that contains the locations of the stained nuclei of all the cells. Further, the detector captures image data associated with cells bearing the first surface label and the second surface label. In one embodiment, each channel provides a separate set of image data. In another embodiment one set of image data is collected with different optical channels associated with each of the nuclear label and two cell type labels. The labels used need not be limited to surface labels and any suitable labels can be used as is known in the fields of biology, chemistry, and physics. While in another embodiment, three sets of image data are collected, one for the nuclear label positions, one for the first label positions, and one for the second label positions.

These images may be captured through various fields of view. For example, in a coculture of stained or labeled cells, one type of cell may fluoresce in the blue spectrum, i.e., in the 490-495 nm wavelength range. Another cell may fluoresce in the red spectrum, i.e., in the 620-750 nm wavelength range. The location of the first cell may be detected by the presence of a spike in the blue wavelength 450-495 nm at the cell's location in the cell array. Accordingly, the second cell may be detected by a spike in the red wavelength (620-750 nm) in the cell array at the location the cell. However, as discussed herein any labeling techniques can be used to identify the nuclei of all of the cells in the coculture and to distinguish all of the cell types present.

The approach described above uses the nuclear stain to determine cell coordinate data coupled with a distance metric to determine two subpopulations of cells that are adjacent or non-adjacent to each other. As a further step, the surface labels discussed above, or any other suitable cell type labels, can be used to identify cells as adjacent or non-adjacent as a function of cell type (Step 106). Thus, in particular it is desirable to identify those cells that are of (1) different cell types, cancerous and non-cancerous for example, and (2) adjacent to each other. This can be achieved by using some of the detectors and image analysis tools discussed herein. From the previous steps, the overall population of adjacent cells is known.

Given the existing labeling of cells of different cell types, the image data associated with each of the labeled cells can be compared against the set of adjacent cells in the coculture to determine a further subset of adjacent cells that are of different cell types. In some embodiments this can be achieved by using nuclear masks that overlay the positional data relative to the surface stains such that the surface stain intensity can be more easily and precisely determined to evaluate cell type. Thus, when using nuclear staining for determining cell position, a nuclear mask can be used that expands a geometric region of pixels around each cell's nucleus such that it extends into cytoplasm. As a result, when imaged the label associate with a particular cell type will appear in the cytoplasm region that extends beyond the nucleus, but within the nuclear mask. This process facilitates cell type identification. An example of identified cell, as a function of cell types is shown in FIG. 5B.

FIG. 2 is a depiction of the nuclear coordinate system and distance metric calculation method of the present invention. This metric can be used a part of (Step 104) discussed above to score each cell as adjacent or non-adjacent in the coculture. The image in FIG. 2 contains two nuclei, a first nucleus 2 and a second nucleus 4. As shown, these two nuclei are at a substantially central position relative to the cell membrane or cell wall. Previously, a first label and a second label were applied to the coculture. Although the nuclear stains cannot distinguish different cell types, as a nuclear stain stains all cells similarly, the cell types can be distinguished based on surface markers. Further, cancerous cells are often polyploid and will absorb more nuclear stain than non-cancerous cells. Thus, the cancerous cells can be distinguished based on the intensity of the nuclear staining. In light of the targeted nuclear labeling, a software controlled imaging system can image the coculture and assign an x coordinate and a y coordinate to each of the nuclei. As an example, position data is shown in Table 1. For example, the first nuclei 2 has an x coordinate x₁ and a y coordinate y₁. The second nucleus 4 has an x coordinate x₂ and a y coordinate y₂.

The distance d between the two nuclei is calculated using these coordinates and the Pythagorean Theorem, where

d=\sqrt{(Δx)²+(Δy)²}

and Δx is the x distance between x₁ and x₂ and Δy is the y distance between y₁ and y₂. If the distance d is less than the average diameter of the cells, the two cells are considered to be adjacent to each other. In contrast, if the distance d is greater than the average diameter of the cells, the two cells are considered to be non-adjacent. FIG. 2 only depicts two cells, however, in implementation, this method can determine if multiple cells, for example hundreds or thousands of cells, are in contact with each other. Although the Pythagorean Theorem is used to provide a first order adjacency calculation based on known average cell diameter values, other distance metrics such as a three dimensional extension of the Pythagorean Theorem or other topological distance metrics can be used.

When examining the effect of one cell's contact with another cell, the present invention determines a group or subset of cells that are (1) in contact with each other and (2) of different cell types. In another embodiment, the techniques disclosed herein can be used when there a population of cells of only a single cell type. This population of cells of the same type can be divided into (1) cells growing in isolation and (2) cells that are growing in contact with a neighboring cell. The gene expressions or factor secretions can be examined for cells growing in isolation, versus those growing in contact. In this embodiment, only a single stain would be needed, i.e., the nuclear stain. Once the appropriate cells have been identified, those cells can be observed and analyzed to determine the affect of one cell upon the other. For example, if one wishes to observe the effect of a cancer cell has on a cell in close proximity to that cancer cell, a researcher can use the present invention to identify cancer cells in contact with other cells. Once those cells have been identified, the researcher can observe the interactions of those specific cells and their response to different compounds. A multi-well plate implementation facilitates such experiments. In one embodiment, when a multi-well plate is used, each well can be centrally imaged as a field, within each field, each nucleus is assigned a coordinate and an annotation as to which well the field originates.

Further, once the endothelial cell population has been divided into epithelial adjacent and nonadjacent subpopulations, measurements collected by high content imaging can be compared between these two populations. This comparison facilitates identifying changes in endothelial cells induced by contact with epithelial cells and vice versa. In turn, to the extent these changes correspond to genetic anomalies, cancer, or other pathologies of interest, their study can be used as part of a pharmaceutical investigation or other research program.

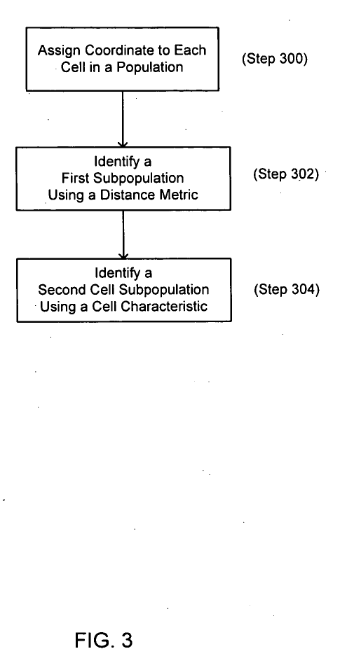

FIG. 3 is a high level flow chart depicting another exemplary embodiment of the present invention. In this embodiment, the first step of the method for determining whether cells are proximate to one another is to assign a coordinate to each cell (Step 300). A coordinate is assigned to each cell once the cells have been stained or labeled and detected with a detector. Then, a first subpopulation is determined using a distance metric (Step 302). The first subpopulation is cells that are in contact with each other. The distance metric can be the Pythagorean Theorem discussed above or other suitable distance metrics. Finally, a second subpopulation is identified using a cell characteristic (Step 304). The cell characteristic may be the type of cell, whether or not the cell is cancerous, the age of the cell, the size of the cell, antibody affinity, receptivity to stain, receptivity to label, light reflectance, fluorescence, light absorption or any other distinguishing feature that may be determined through the staining of the cells and the subsequent detection and analysis of the cells by a software program.

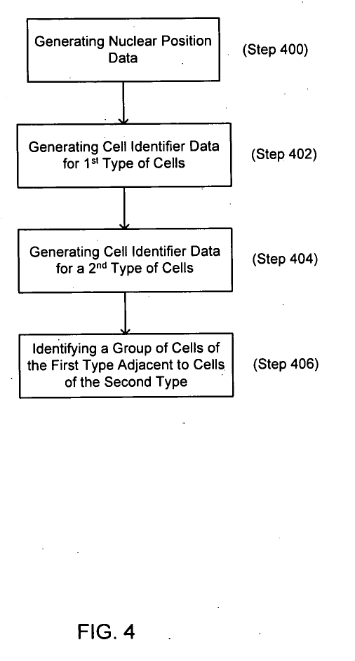

FIG. 4 is a high level flow chart depicting another exemplary embodiment of the present invention. In this embodiment, nuclear position data is generated (Step 400). The nuclear position data is generated through the use of a detection mechanism to detect the location of the previously stained cells. Then, cell identifier data is generated for the first type of cells (Step 402). Then, cell identifier data is generated for the second type of cells (Step 404). The cell identifier data typically includes the type of cell and is generated in response to light or another signal originating from a labeled cell. Finally, a group of cells of the first type are identified as in contact with a group of cells of a second type (Step 406). The cells are identified as in contact with another cell if they meet suitable distance metric criteria as discussed above.

### EXAMPLES

In light of the discussion provided above, some specific experimental examples are considered below. It should be understood that the above-described embodiments and the following examples are meant to illustrate, not limit, the scope of the present invention.

### Example 1

In one exemplary experiment, cocultures of endothelial and cancer epithelial cells were prepared in multi-well plates. After a period of extended culture (24-96 hours or longer), the cells were washed and fixed using 0.4% paraformaldehyde. Fixed cells were incubated with antibodies that recognize an endothelial cell specific marker, CD44, as well an epithelial cell specific marker, CDW75. These primary antibodies are derived from different species host (e.g. mouse and goat) so that they may then be detected with species specific secondary antibodies labeled with different fluorescent molecules (e.g. Alexa 488 and Alexa 594). Nuclear DNA in the cells of the cocultures was stained with DAPI.

Stained cocultures were placed on the stage of an inverted fluorescent microscope equipped with a CCD camera. Three images were captured for each field of view. The first image obtained was of the DAPI DNA stained nuclei, as shown in FIG. 5A. Thus, FIG. 5A is a depiction of a coculture of cells stained with DAPI. As seen in FIG. 5A, DAPI specifically binds to double stranded DNA, and when excited with light, the DAPI-DNA complex fluoresces a bright blue color, permitting the detection of the cells of interest. Another image was obtained (not shown), of the CD44 stained endothelial cells. A third image was obtained of the CDW75 stained LNCaP cancer epithelial cells, as shown in FIG. 5B. The images were captured from multiple fields of view. Each of the fields of view was tracked using annotations in the data file. Other stains, such as membrane stains, metabolic stains, fluors conjugated to an antibody or binding protein, or any stain commercially available in Invitrogen Corp.'s (Carlsbad, Calif.) Molecular Probes catalog can be used.

Image analysis software, such as Cellomics' Arrayscan Bioapplication or the open source CellProfiler image analysis software was used to identify and outline the nuclei of all cells using the techniques discussed above. Also, the CellProfiler software was used to measure the intensity of the cell type labels.

As discussed above, a nuclear mask based approach was issued to enhance detection of the cell type specific labels. The nuclear masks were dilated by about two pixels and applied to the images of the CD44 and CDW75 stained cells. Each nuclear mask identifies the location of a cell within the CD44 and CDW75 images. Within the area of the mask, the intensity of staining of CDW75 or CD44 can be quantified and used to identify a cell as either epithelial or endothelial.

High Content Image Analysis Software provided the x-y coordinate location of a nucleus within a given image. Using the nuclear x-y coordinates, the x and y offsets of one cell from another cell can be calculated. These offsets provide the length of the sides of a right triangle (shown in FIG. 2). Using the Pythagorean Theorem, the hypotenuse distance between the two nuclei can be calculated. If the nuclear distance was less than the average cell diameter, the two nuclei are considered to be adjacent cells in contact. As a result, using this distance metric for all of the cells in the coculture, a scoring and a map of all adjacent cells can be determined. From the total scoring of all adjacent cells, the application of the labels can be used to identify the subset of cells that (1) adjacent and (2) of different cell types.

The nuclear distance, the CDW75 staining, and the CD44 staining were then used to determine which endothelial cells were in contact with an epithelial cell through the following process. The first step includes identifying all cells in the overall coculture population using the nuclear stain DAPI. The second step is to subdivide the population of all cells into two subpopulations, those cells that stain with CD44 (endothelial) and those that stain with CD75 (cancer epithelial). Finally, the third step is to further subdivide the endothelial population into two subpopulations, those endothelial cells adjacent to a cancer epithelial cell and those non-adjacent to an epithelial cell. A list of all epithelial cells (CD75 positive) and endothelial cell (CD44 positive) was constructed. The Pythagorean distance for each epithelial cell compared to each endothelial cell was calculated. The distances smaller than the average diameter of an epithelial cell were flagged as endothelial cells adjacent to epithelial cells.

The result of the process is shown in FIG. 5C, a scatter plot of the x-y coordinates of each cell in the cell coculture. The plots are useful to explain the process or validate that an algorithm is working. As shown in FIG. 5C, a coculture population of Human Umbilical Vein Endothelial Cells, (HUVEC cells) cells are marked as circles, cancer epithelial cells are marked as Xs and endothelial cells adjacent to a cancer epithelial cell are marked as triangles. From the scatter plot, one can quickly determine cells of interest, i.e. cells in contact with cancer cells. Cell data also can be analyzed in table format. For cell data in a table, each cell is annotated, the cells of the desired type can be sorted and their responses averaged algorithmically. These cells of interest can then be monitored and studied to further understand the interaction between cancer cells and healthy cells. For example, a temporal analysis of different cancer/non-cancer cell pairs can show when a cancer cell dies and the other cells lives in response to a particular treatment compound, such as an anti-vascularization candidate drug.

### Example 2

In one preferred embodiment, a plurality of cocultures are prepared using a multi-plate, with one coculture per well, to facilitate batch processing and the testing of multiple experimental agents such as drug candidates or siRNAS. Plate endothelial cells (such as HUVEC cells) were deposited into a multi-well plate. LNCaP cells are androgen-sensitive human prostate carcinoma cells. As part of this example, the cancerous epithelial LNCaP cells were transfected with siRNA targeting genes of interest that have been identified using expression profiling as being modulated in LNCaP cells upon contact with HUVEC cells also were deposited into a multi-well plate. The genes of interest potentially will be new targets for the development of small molecule inhibitors. The combination was then cultured for 48 hours.

The plate was then processed. First, the cells were fixed and stained with DAPI (nuclear stain), CDW75 (stains LNCaP specifically) and a marker protein i.e. an antibody that recognized PARP, a marker for apoptosis. The marker protein can mark cell behavior of interest, such as apoptosis or activation of certain pathways. Then, the plate was scanned using automated image capture (Cellomics ArrayScan VTi). The results of the scan were superimposible images of all three stains for multiple fields within a well. The image was then analyzed.

A workflow for obtaining information with respect to cell type and cell position was designed using an optical system. In one embodiment, the CellProfiler image analysis software was used to obtain the relevant cell data from a coculture. One embodiment of the method is as follows. For each well: (1) identify each field, then (a) identify each cell using the nuclear stain DAPI (b) measure the intensity of CDW75 or CD44 for each cell and (c) record x, y coordinates of each cell, (2) run the same process of Step 1 for the next field, (3) run the whole process of Steps 1 and 2 on the next well. Finally, all of the data was outputted to a spreadsheet database, for example Microsoft Excel (Microsoft, Inc. Redmond, Wash.). A subset of such a database representation is shown in Table 1. As discussed above, the data Table 1 can be formatted such that is can be operated on using a script or other code portion, such as an R script. As shown in the table, gene abundance data is also captured.

Once the data was outputted to Microsoft Excel, it was analyzed. A column was created that flagged all LNCaP cells (i.e., CDW75 staining is >0.12 Relative Fluorescent Units (“RFU”)). Then a list was made of all wells and fields. Then, the following processing sequence loop was run through for all the wells and fields. First, within a field loop (1) generate a list of LNCaP cells (cells that stained with CDW75) (2) generate a list of HUVEC cells (cells that did not stain with CDW75) (3) compare each HUVEC cell to the LNCaP list of cells, based on nuclei distance. If the distance between the nuclei was less than a cell diameter, the HUVEC cell was flagged as an LNCaP neighbor, otherwise it was flagged as a non-neighbor. Then the annotated data file was written, which had all cells labeled as either LNCaP, HUVEC, or HUVEC neighbor.

Then, the genes that have been knocked down by siRNA are identified in a list. For each gene, (1) the data was pulled out for that gene (2) data set was separated into 2 populations, HUVEC_nonneighbor, and HUVEC neighbor (3) the populations were compared with a T-test. As is known in statistics, a T-test is used to evaluate whether the means of two groups are statistically different from each other. Thus, in this instance the T-test is used to determine if the staining intensity of the marker gene are statistically different between the two populations. As part of this process flow, (4) the p value was appended for the t-test to the data set. As is known from the statistical arts, the p-value is a measure of consistency between experimental results obtained using the approaches discussed herein in a given trial and the “pure chance” explanation for those results. Next, the data set was written to a file, which was opened with visualization software such as Spotfire (Spotfire, U.S., 212 Elm Street, Somerville, Mass. 02144). Finally, the p values vs. genes of interest were plotted. The genes of interest were examined that differentially affect the expression of the marker protein being measured by high content screening.

TABLE 1

| well | X | y | GOI | CDW75 | Reagent | Gene | Incap | adjacent | cell |
|---|---|---|---|---|---|---|---|---|---|
| C01 | 20.4366 | 266.383 | 0.187079 | 0.157253 | ScrV_SM | ScrV | TRUE | TRUE | LNCaP |
| C01 | 44.9619 | 138.263 | 0.180515 | 0.131658 | ScrV_SM | ScrV | TRUE | TRUE | LNCaP |
| C01 | 70.8828 | 359.803 | 0.175014 | 0.103778 | ScrV_SM | ScrV | FALSE | FALSE | HUVEC |
| C01 | 74.8595 | 414.908 | 0.164087 | 0.09776 | ScrV_SM | ScrV | FALSE | FALSE | HUVEC |
| C01 | 96.8484 | 22.6338 | 0.1737 | 0.112755 | ScrV_SM | ScrV | FALSE | FALSE | HUVEC |
| C01 | 155.672 | 287.22 | 0.205842 | 0.239115 | ScrV_SM | ScrV | TRUE | TRUE | LNCaP |
| C01 | 150.144 | 268.453 | 0.196136 | 0.196756 | ScrV_SM | ScrV | TRUE | TRUE | LNCaP |
| C01 | 166.615 | 235.058 | 0.180796 | 0.107451 | ScrV_SM | ScrV | FALSE | FALSE | HUVEC |
| C01 | 174.363 | 335.672 | 0.184684 | 0.107505 | ScrV_SM | ScrV | FALSE | FALSE | HUVEC |
| C01 | 195.522 | 275.466 | 0.200649 | 0.16777 | ScrV_SM | ScrV | TRUE | TRUE | LNCaP |
| C01 | 199.486 | 167.175 | 0.199371 | 0.206136 | ScrV_SM | ScrV | TRUE | TRUE | LNCaP |
| C01 | 199.841 | 143.514 | 0.220899 | 0.26885 | ScrV_SM | ScrV | TRUE | TRUE | LNCaP |
| C01 | 214.872 | 283.423 | 0.20832 | 0.185849 | ScrV_SM | ScrV | TRUE | TRUE | LNCaP |

TABLE 2
(Exemplary R Script Embodiment)

```
rm(list=ls(all=TRUE))
library(lattice)
t1 <- Sys.time( )
working.dir <- “b:\\public\\CellProfiler\\jz060728\\pH3A”
file.prefix <- “JZ060728”
file.name.nuclei<- paste( working.dir,“/”,file.prefix, “_ExpandedNuclei.txt”, sep=“”)
#file.name.huvec<- paste( working.dir,“/”,file.prefix, “_HUVEC.txt”, sep=“”)
#file.name.lncap<- paste( working.dir,“/”,file.prefix, “_LNCaP.txt”, sep=“”)
out.file <- paste( working.dir,“/”,file.prefix, “_out.txt”, sep=“”)
#file.name.nuclei<- paste( working.dir,“\\”,file.prefix, “_ExpandedNuclei.txt”, sep=“”)
#file.name.huvec<- paste( working.dir,“\\”,file.prefix, “_HUVEC.txt”, sep=“”)
#file.name.lncap<- paste( working.dir,“\\”,file.prefix, “_LNCaP.txt”, sep=“”)
#out.file <- paste( working.dir,“\\”,file.prefix, “_out.txt”, sep=“”)
#map.file <- paste( working.dir,“\\”, “96map.txt”, sep=“”)
map.file <- paste( working.dir,“/”, “96map.txt”, sep=“”)
map <- read.table (file = map.file, n = −1, sep = “\t”, dec = “.”, header=TRUE, skip = 0,
na.strings = “NA”, strip.white = FALSE )
d <- read.table (file = file.name.nuclei, n = −1, sep = “\t”, dec = “.”, header=TRUE, skip = 2,
na.strings = “NA”, strip.white = FALSE )
d <- d[ d$CenterX..ExpandedNuclei..Location != 0,]
tempname <- “null”
imagenames<-d$X
holder<- character( length( imagenames ) )
for( i in 1:length( imagenames )){
  if( imagenames[i]!=“” ){
  holder[i] <- strsplit( as.character(imagenames[i] ), “AS_VTI_Z4_”, extended=FALSE)
[[1]][2]
    tempname<- holder[i]
  }else{
  holder[i] <- tempname
  }
}
well <- substring( holder, 14, 16 )
field <- substring( holder, 18, 19 )
mody <- 512 - d[4]
d2 <- cbind( holder, well, field, d[ ,c( 3,4,6,17) ], mody )
names(d2)<-c(“name”,“well”,“field”,“x”,“y”,“GOI”,“CDW75”,“mody”)
d3 <- merge( d2, map, by.x=“well”, by.y=“Well” )
#used 0.15 for STAT3
#used 0.12 for Eph2B
lncap <- d3$CDW75 > 0.12
d3 <- cbind( d3, lncap )
rm( lncap, holder, d, d2, map, mody, well, field, imagenames )
##############################################
#d2 <- d2[ d2$well==“B01” & d2$field==“01”, ]
#d2.mcf7n <- d2.mcf7n[ d2.mcf7n$well==“B01” & d2.mcf7n$field==“01”, ]
##########################################################
#How many wells to analyze?
wells <- unique( d3$well)
num.wells <- length( wells )
fields <- unique( d3$field )
num.fields <- length( fields )
celldiam <- 35
cell.same <- 10
firsttime<-TRUE
# k<-1; l<-1
for( k in 1:num.wells ){
for( l in 1:num.fields ){
cells.field <- d3[d3$well== wells[k] & d3$field== fields[l], ]
#pull out the x,y locations of mcf7 cells
#lncap.cells <- d3.lncap[d3.lncap$well== wells[k] & d3.lncap$field== fields[l],]
lncap.cells <- d3[d3$well== wells[k] & d3$field== fields[l] & d3$lncap== TRUE,]
num.cells <- nrow( cells.field )
num.lncap <- nrow( lncap.cells )
if( num.lncap > 0 & num.cells > 0 ){
    adjacent <- vector( mode=“logical”, length=num.cells)
    LNCaP <- vector( mode=“logical”, length=num.cells)
    # i<-14; j<-1
    for( i in 1:num.cells ){
    for( j in 1:num.lncap ){
    if( sqrt(( cells.field[i,]$x − lncap.cells[j,]$x )\hat{ }2 +( cells.field[i,]$y − lncap.cells[j,]$y )\hat{ }2)
< cell.same){
    LNCaP[i] <- “TRUE”
    }
    if( sqrt(( cells.field[i,]$x − lncap.cells[j,]$x )\hat{ }2 +( cells.field[i,]$y − lncap.cells[j,]$y )\hat{ }2)
< celldiam ){
    adjacent[i] <- “TRUE”
    }
    }
    }
    cell <- paste( adjacent, LNCaP, sep=“_”)
    cell[cell==“TRUE_TRUE”] <- “LNCaP”
    cell[cell==“TRUE_FALSE”] <- “adjacentHUVEC”
    cell[cell==“FALSE_FALSE”] <- “HUVEC”
    #cell <- lncap.adjacent
    #cell[cell==“TRUE_TRUE”] <- “LNCaP”
    #cell[cell==“FALSE_TRUE”] <- “adjacentHUVEC”
    #cell[cell==“FALSE_FALSE”] <- “HUVEC”
    #d4 <- cbind( d4, lncap.adjacent, cell )
    d4 <- cbind( cells.field, adjacent, LNCaP, cell )
    if( !firsttime ) holder <- rbind( holder, d4 )
        if( firsttime ){
            holder<- d4
            firsttime<-FALSE
        }
    }#if lncap > 0 loop
} #fields loop
} #wells loop
write.table( holder, file = out.file, append = FALSE, quote = FALSE, sep = “\t”, eol = “\n”, na =
“NA”, dec = “.”, row.names = FALSE, col.names = TRUE, qmethod = c(“escape”, “double”))
rm( d )
d <- holder
genes <- unique( d$Gene )
genes <- genes[ genes != “Mock” ]
num.genes <- length( genes )
firsttime<-TRUE
# i <- 1
for( i in 1:num.genes ){
    gene.data <- d[ d$Gene == genes[i],]
    huvec <- gene.data[ gene.data$cell == “HUVEC”,]
    adjhuvec <- gene.data[ gene.data$cell == “adjacentHUVEC”,]
    mean.huvec <- mean( huvec$GOI, na.rm=TRUE)
    sd.huvec <- sd( huvec$GOI, na.rm=TRUE)
    mean.adjhuvec <- mean( adjhuvec$GOI, na.rm=TRUE)
    sd.adjhuvec <- sd( adjhuvec$GOI, na.rm=TRUE)
    p <- t.test( log2(huvec[ !is.infinite( huvec$GOI),]$GOI), log2( adjhuvec[
!is.infinite(adjhuvec$GOI),]$GOI ))[[3]][1]
# t.test( huvec[ !is.infinite( huvec$GOI),]$GOI, adjhuvec[ !is.infinite(adjhuvec$GOI),]$GOI )
    results <- data.frame( genes[i], mean.huvec, sd.huvec, mean.adjhuvec, sd.adjhuvec, p )
if( !firsttime ) holder<-rbind( holder, results)
if( firsttime ){
    holder<- results
    firsttime<- FALSE
    }
}
results.file <- paste( working.dir,“\\”,file.prefix, “_results.txt”, sep=“”)
write.table( results.final, file = results.file, append = FALSE, quote = FALSE, sep = “\t”, eol =
“\n”, na = “NA”, dec = “.”, row.names = FALSE, col.names = TRUE, qmethod = c(“escape”,
“double”))
t2 <- Sys.time( )
elapsed <- t2−t1
```

### EQUIVALENTS AND SCOPE

Those skilled in the art will recognize, or be able to ascertain using no more than routine experimentation, many equivalents to the specific embodiments, described herein. The scope of the present invention is not intended to be limited to the above Description, but rather is as set forth in the appended claims.

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention can be excluded from any one or more claims, for any reason, whether or not related to the existence of prior art.

### INCORPORATION OF REFERENCES

All publications and patent documents cited in this application are incorporated by reference in their entirety to the same extent as if the contents of each individual publication or patent document were incorporated herein.

What is claimed is:

1. A method of identifying, in a coculture of cells of differing cell types, cells of a first cell type in contact with cells of a second cell type, the method comprising the steps of:
a) distinguishing cells of a first cell type from cells of a second cell type; and
b) identifying cells of a first cell type in contact with cells of a second cell type.

2. The method of claim 1, wherein step a) comprises:
(i) labeling the cells of a first cell type with a first label, thereby to permit the distinguishing of the cells of the first cell type from the cells of the second cell type; and
(ii) detecting the first label, thereby to identify cells of the first cell type.

3. The method of claim 2, wherein step a) further comprises:
(iii) labeling the cells of the second cell type, but not the first cell type, with a second label; and
(iv) detecting the second label, thereby to facilitate distinguishing the cells of the first cell type from the cells of the second cell type.

4. The method of claim 3, wherein step b) comprises identifying cells labeled with the first label adjacent cells labeled with the second label.

5. The method of claim 3, wherein the second label is a labeled anti-CD44 antibody.

6. The method of claim 1, wherein step b) comprises:
(i) labeling the nuclei of the cells of the first cell type and the cells of the second cell type with a nuclear label;
(ii) detecting the nuclear label, thereby to detect the locations of the nuclei in the coculture; and
(iii) identifying cells whose internuclear distances are less than or equal to approximately one cell diameter, thereby to identify cells in contact with each other.

7. The method of claim 6, wherein step b) further comprises calculating distances between nuclei in the coculture.

8. The method of claim 1, wherein the first cell type is an endothelial cell and the second cell type is an epithelial cell.

9. The method of claim 8, wherein step a) comprises the steps of:
(i) labeling the epithelial cells, but not the endothelial cells, with a labeled anti-CDW 75 antibody; and
(ii) detecting the labeled anti-CDW75 antibody.

10. A method of identifying a cancer epithelial cell, the method comprising the steps of:
a) exposing the cancer epithelial cell to an anti-CDW75 antibody; and
b) detecting the anti-CDW75 antibody, thereby to identify the cancer epithelial cell.

11. A method of identifying subpopulations of cells within a population of cells, the method comprising the steps of:
assigning a spatial coordinate to each cell in the population, the spatial coordinate identifying a relative position of each cell;
identifying a first subpopulation of the population using a distance metric, each cell of the first subpopulation substantially adjacent to another cell in the population; and
identifying a second subpopulation of the first subpopulation using a cell characteristic, each cell of the second subpopulation substantially adjacent to a cell of interest, the cell of interest having a cell type.

12. The method of claim 11, wherein the distance metric relates two spatial coordinates and cell diameter data to determine cell adjacency.

13. The method of claim 11 wherein the cell characteristic is selected from the group consisting of antibody affinity, receptivity to a stain, receptivity to a label, light reflectance, fluorescence, and light absorption.

14. The method of claim 11 wherein the cell type is selected from the group consisting of endothelial, epithelial, stem, and cancer.

15. A method of identifying cell adjacency in a coculture of cells, the method comprising the steps of:
generating a first set of image data of the coculture of cells, the first set of image data comprising nuclear position data for each imaged cell;
generating a second set of image data of at least a subset of the coculture of cells, the second set of image data comprising cell identifier data associated with a first cell type;
generating a third set of image data of at least a subset of the coculture of cells, the third set of image data comprising cell identifier data associated with a second cell type; and
identifying a group of cells in response to the nuclear position data, wherein each cell having the first cell type in the group is adjacent another cell in the group having the second cell type.

16. The method of claim 15 wherein the coculture of cells is disposed in a well of a multi-well plate, chamber slide, microscope slide, or Petri dish.

17. The method of claim 15 wherein the first cell type is endothelial and the second cell type is epithelial.

18. The method of claim 15 wherein the cell identifier data is generated in response to light originating from a labeled cell.
